(19) United States
(12) Patent Application Publication
(10) Pub. No.: US 2020/0390878 A1
(43) Pub. Date: Dec. 17, 2020

(54) RECOMBINANT SWINEPOX VIRUS AND VACCINES

(71) Applicant: CEVA SANTE ANIMALE, LIBOURNE (FR)

(72) Inventors: DOLORES RODRIGUEZ AGUIRRE, CANTOBLACO - MADRID (ES); MATEO DEL POZO VEGAS, VALDEOLMOS - MADRID (ES); JOSE FRANCISCO RODRIGUEZ AGUIRRE, CANTOBLACO - MADRID (ES); JUAN RAMON RODRIGUEZ FERNANDER-ALBA, VALDEOLMOS - MADRID (ES)

(21) Appl. No.: 16/770,636

(22) PCT Filed: Dec. 7, 2018
(86) PCT No.: PCT/EP2018/084033

§ 371 Date: Jun. 8, 2020

(30) Foreign Application Priority Data
Dec. 8, 2017 (EP) 17306728.1

(51) Int. Cl. A61K 39/12; C12N 15/86

(52) U.S. Cl. CPC A61K 39/12; C12N 15/86; A61K 2039/5256; C12N 2750/10034; C12N 2710/24043

(57) ABSTRACT

The present invention relates to novel recombinant swinepox viruses and their use in vaccine compositions. The recombinant swinepox viruses of the invention contain an inactive serpin gene and may be used to express any nucleic acid of interest.

The present invention relates to novel swinepox viruses and their use. The swinepox viruses of the invention contain an inactive serpin gene of swinepox virus and may incorporate one or several foreign gene sequences. The SPV of the invention are particularly suited to produce swine vaccines, particularly for vaccinating against PCV2 infection.

### BACKGROUND

Different types of viruses have been proposed in the art as vector for gene delivery or peptide expression in vivo. In particular, veterinary vaccines have been prepared that express at least one relevant antigen using recombinant viruses such as poxviruses (Ogawa R. et al., Vaccine, 8:486-490 (1990)), adenoviruses (HSU, K. H. et al., Vaccine, 12; 607-612 (1994)), baculoviruses, as well as herpesviruses (Shin, M.-F. et al., Proc. Natl. Acad. Sci. U.S.A., 81:5867-5870 (1984)). Examples of specific virus vectors that permit the expression of a gene for a foreign antigen include Aujeszky's disease virus (pseudorabies virus; PRV) (Van Zijl M. et al., J. Virol., 65:2761-2765 (1991)), herpesvirus of turkey (HVT) (Morgan R. W. et al., Avian Dis. 36:858-870 (1992)), and Marek's disease virus (MDV). Recombinant vectors based on the genus herpesvirus are under intensive study.

There is, however, a need in the art for new viral vector products that can be used to express recombinant peptides or proteins in vivo. In this regard, poxviruses have been engineered to encode different polypeptides. Poxviruses, once released into the blood from infected cells, can infect other cells and thereby potentially lead to elevated expression levels. Recombinant poxviruses have been produced from different types of poxviruses, including cowpox virus, vaccinia virus, and swinepox virus (SPV). SPV recombinants have been produced by cloning foreign gene sequences in the TK gene or in the IL18bp gene (Richard W. Moyer, Eladio Vinuela, E. P. J. Gibbs, U.S. Pat. No. 5,651,972, WO2016/097281).

The invention now proposes novel rSPV comprising an inactivated serpin gene. As shown in the present application, such deletion does not alter recombinant virus growth or expression of recombinant genes, can increase cloning capacity, and further attenuates the recombinant virus. The resulting recombinant SPV viruses allow efficient and stable expression of cloned gene sequences and can be used to produce therapeutics or vaccines for treatment of any mammal, particularly swine.

### SUMMARY OF THE INVENTION

The present invention relates to a recombinant swinepox virus (rSPV) comprising an inactive serpin gene. In a preferred embodiment, the rSPV comprises an inactive serpin gene and an inactive IL18bp gene. In another preferred embodiment, the rSPV comprises an inactive serpin gene and an inactive TK gene. In a further preferred embodiment, the rSPV comprises an inactive serpin gene, an inactive IL18bp gene, and an inactive TK gene.

The invention also relates to the use of a rSPV as defined above for delivery and expression in vivo of a foreign gene.

The invention further relates to a recombinant swinepox virus comprising an inactive serpin gene and containing one or more foreign nucleic acid sequences. The foreign nucleic acid sequence(s) may be inserted in the inactive serpin gene, or in any other cloning site of the virus genome, such as into the IL-18 binding protein (IL18bp) gene, into the thymidine kinase (TK) gene, and/or into the Ankirin repeat protein (ARP) gene. In a particular embodiment, the rSPV comprises an inactive serpin gene and at least one foreign nucleic acid sequence inserted in the viral IL18bp gene sequence, preferably in replacement of all or part of the IL18bp gene.

In a particular embodiment, the invention relates to a recombinant swinepox virus comprising an inactive serpin gene, an inactive IL18bp gene, and an inactive TK gene, and further containing one or more foreign nucleic acid sequences.

A further object of the invention resides in a nucleic acid molecule comprising the genome of a SPV as defined above.

A further object of the invention is a host cell comprising a SPV or a nucleic acid molecule of the invention.

The present invention further provides a method for producing a rSPV, comprising infecting or introducing into a competent cell a nucleic acid molecule as defined above and collecting the rSPV.

The invention also relates to a method for propagating a rSPV, comprising infecting a competent cell with a rSPV as defined above and collecting the rSPV produced by said cells.

The invention also concerns a composition, preferably a veterinary composition, comprising a rSPV as defined above, or a cell as defined above, or a nucleic acid molecule as defined above, and an excipient.

A further object of the invention is a vaccine composition comprising a rSPV as defined above, or a cell as defined above, or a nucleic acid molecule as defined above, a suitable excipient and, optionally, an adjuvant.

The invention also relates to a rSPV or cell or nucleic acid molecule as defined above for use for delivering a therapeutic or vaccinating peptide or protein to a porcine.

The invention also relates to a rSPV or cell or nucleic acid molecule as defined above for use for immunizing or vaccinating a porcine against a pathogen.

The invention also relates to a method of vaccinating a mammal comprising administering to the mammal an attenuated or recombinant SPV of the invention.

The invention also concerns a vaccination kit for immunizing a porcine which comprises the following components:
a. an effective amount of a rSPV or vaccine as defined above, and
b. a means for administering said rSPV or vaccine to said porcine.

A further object of the invention relates to a shuttle plasmid or vector comprising a transgene flanked by two nucleic acid sequences homologous to a serpin gene sequence, said flanking sequences allowing homologous recombination between the shuttle plasmid and a SPV genome.

The invention may be used to deliver and express any foreign gene sequence to a mammal, particularly a porcine. It is particularly suited for expressing foreign antigens (such as PCV2 proteins) to immunize or vaccinate porcine (e.g., pigs, piglets, sow).

### LEGEND TO THE FIGURES

FIG. 1. Genomic structure of wtSPV and rSPVs of the invention

FIG. 2. Genomic structure of rSPVs of the invention

FIG. 3. SPV insertion plasmids

FIG. 4. SPV insertion plasmids

FIG. 5. Expression of PCV2 CAP by a rSPV: the Cap protein presents a nuclear localization.

FIG. 6. EM image of ESK-4 cells infected by rSPV-PCV2-CAP: CAP protein expressed by rSPV self-assembles into virus like particles (VLPs).

### DETAILED DESCRIPTION OF THE INVENTION

The present invention relates to the novel recombinant swinepox viruses and the uses thereof. The viruses of the invention have an inactive serpin gene and may contain one or several foreign nucleic acid sequences, e.g. genes encoding, antigenic or immunostimulatory molecules. The rSPV of the invention may be used to prepare vaccines suitable for treating permissive mammals such as pigs.

Recombinant SPVs

Within the context of the invention, a recombinant swinepox virus designates, generally, a swinepox virus having an artificially (e.g., recombinantly) engineered genome. rSPV include, particularly, swinepox viruses containing one or more genetic deletion(s) and/or foreign genetic material or sequence in their genome. rSPV of the invention typically comprise a SPV genome containing an inactive viral gene and a foreign genetic sequence, packaged into a SPV capsid or envelop, which may also contain a foreign protein or peptide.

rSPVs of the present invention may be prepared or obtained starting from any SPV, such as naturally occurring SPVs, or SPVs available from collections such as ATCC, CNCM, etc, or starting from recombinant SPVs. Preferably, the SPV of the invention are produced or derived from SPV kasza strain (VR-363), isolate 17077-99 (GeneBank Acc: AF410153.1), or strain VTCC/AVA/121 (GeneBank Acc: KJ725378.1). Such SPVs are available from collections or libraries, or may be cloned from their publicly available genomic sequences. Further SPV isolates may also be isolated from infected animals and used to prepare SPV of the invention.

SPVs or rSPV may be cultured or maintained or propagated in any suitable cell. For instance, rSPVs may be cultured, maintained or propagated in embryonic swine kidney cells, such as ESK-4 cells (CL-184), routinely cultured at 37° C. in 5% CO2 in Ham's F-12K medium (Gibco, Cat. No.: 21127-022) supplemented with 1% streptomycin-penicillin (Gibco, Cat. No.: 15140-122) and 5% FBS (Gibco, Cat. No.: 10437-028).

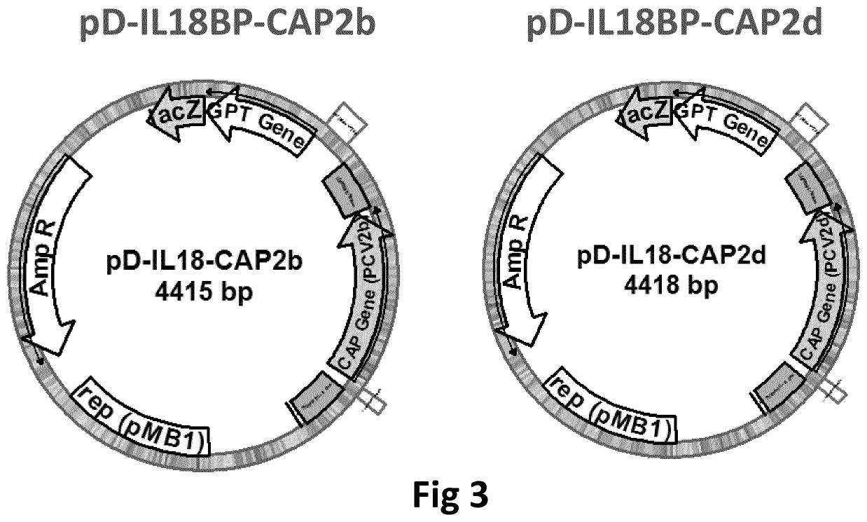

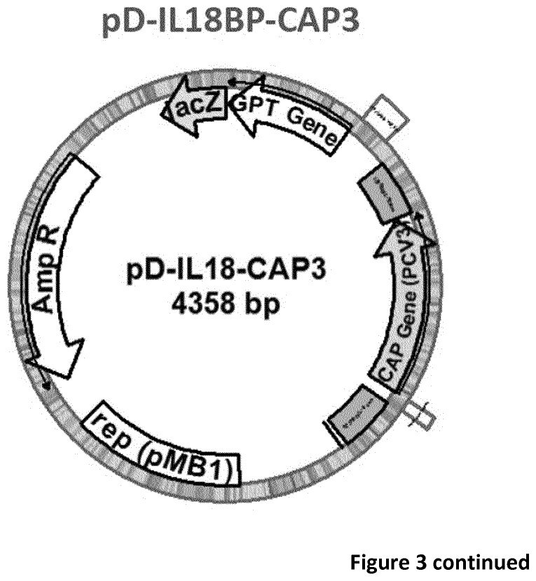

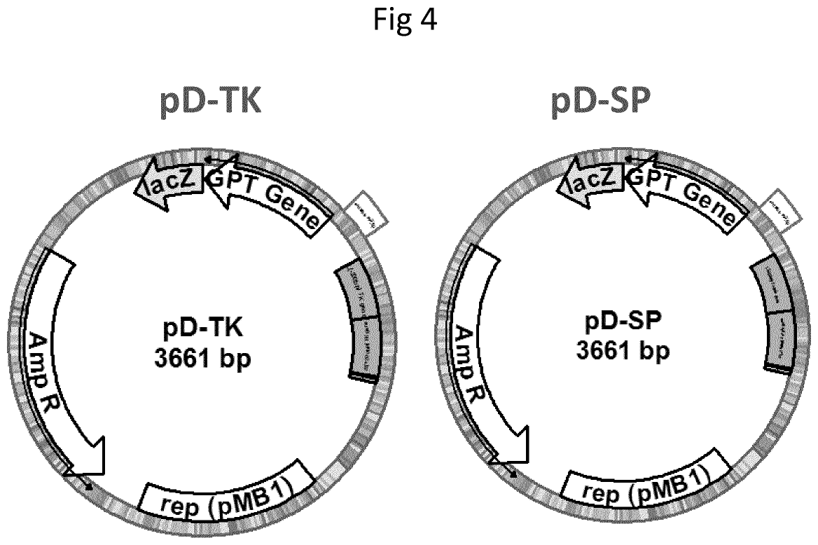

In order to construct a recombinant virus of the present invention, initially, a SPV virus (wt or recombinant) may be propagated in a suitable host cell and the SPV genomic DNA obtained. DNA can be extracted from virus-infected cells according to any conventional method. For instance, cells grown in monolayers can be scraped and then spun to harvest the supernatant. After protein is denatured in a lysis buffer and removed, DNA can be extracted with phenol and/or ethanol. Subsequently, the serpin gene region of the genomic DNA is inactivated (e.g., by mutation, deletion and/or insertion into the serpin coding sequence, for instance). Optionally, further modification(s) of the viral genome can be made, such as insertion of a foreign nucleic acid sequence (or a cloning site allowing insertion of a foreign gene sequence), inactivation of other viral genes, etc. The recombinant SPV genome thus obtained may be used to produce rSPV by transformation of suitable competent cells according to conventional techniques and collection of rSPVs. Alternatively, a shuttle vector may be produced containing a foreign nucleic acid sequence (or a cloning site) flanked by sequences homologous to a serpin gene regions (see e.g., insertion plasmids of FIG. 3-4). Upon introduction into a competent cell in the presence of a SPV virus or genome, homologous recombination between the shuttle vector and the genome generates a rSPV containing an inactive serpin gene. Of course, once a rSPV has been engineered as described above, it can be easily replicated and propagated by simple culture on any competent cells.

Within the context of the invention, an “inactive” (or defective) gene designates a gene which has been modified and is unable to encode a functional wild-type protein or RNA encoded by the wild type gene. The gene is typically inactive as a result of a genetic alteration in the gene sequence, preferably the promoter or coding sequence, most preferably in the coding sequence. The genetic alteration may be a substitution (point mutation), addition and/or insertion of one or more nucleotides in the (coding) sequence, resulting in a reduced or total incapacity of the gene to encode a functional wild type protein/RNA. Typically, an inactive gene is a partially or fully deleted gene, typically containing a deletion of at least 20 bp within a coding sequence of said gene, preferably at least 50 bp, even more preferably at least 100 bp, further more preferably at least 150 bp, at least 200 bp, or even at least 300 bp. In a particular embodiment, the gene is inactive as a result of a full deletion of the coding sequence.

The serpin (or Serine Protease Inhibitor) gene of a viral SPV DNA contains approximately 960 bp and is generally located at nt residues 141494-142456 of a SPV genome. As a specific example, in SPV kasza strain (VR-363), the serpin gene is located at nt141494-142456. The exact position of the serpin gene in any strain of SPV may be identified easily using common knowledge, routine sequence analysis and/or sequence alignment.

Preferred rSPVs of the invention comprise an inactive serpin gene, wherein the endogenous serpin gene lacks (has been deleted of) at least 50 nt, preferably at least 100 nt, even more preferably at least 150 nt, at least 200 nt, at least 250 nt, at least 300 nt, at least 400 nt, at least 500 nt, at least 600 nt, at least 700 nt, at least 800 nt, further more preferably between 400 nt and 950 nt in the gene sequence, preferably in the coding sequence. Specific and preferred rSPVs of the invention contain a deletion of at least nt400-600 of serpin gene, even more preferably of at least nt300-700 of serpin gene, such as nt200-800 of a serpin gene. The invention shows such rSPVs can be stably propagated, have an increased cloning capacity, and have a reduced virulence.

In a particular embodiment, the rSPVs of the invention contain an inactive serpin gene and a further inactive gene selected from IL18bp, TK and ARP. In a preferred embodiment, the rSPV contains an inactive serpin gene and an inactive IL18bp gene. In another preferred embodiment, the rSPV contains an inactive serpin gene and an inactive TK gene. In a particularly preferred embodiment, the rSPV contains an inactive serpin gene, an inactive IL18bp gene, and an inactive TK gene.

The IL18bp gene of a viral SPV DNA contains approximately 402 bp, and is generally located at nt residues 7745-8146 of a SPV genome. As a specific example, in SPV kasza strain (VR-363), the IL18bp gene is located at nt7745-8146. The exact position of the IL18bp gene in any strain of SPV may be identified easily using common knowledge, routine sequence analysis and/or sequence alignment. Preferred rSPVs of the invention comprise an inactive IL18bp gene, wherein the endogenous IL18bp gene lacks at least 50 nt, preferably at least 100 nt, even more preferably at least 150 nt, at least 200 nt, at least 250 nt, at least 300 nt, further more preferably between 320 nt and 380 nt. Specific and preferred rSPVs of the invention contain a deletion of at least nt 100-200 of IL18bp gene, even more preferably of at least nt50-300 of IL18bp gene, such as nt31-382 or nt19-369 of IL18bp gene.

The TK gene of a viral SPV DNA contains approximately 543 bp, and is generally located at nt residues 55625-56167 of a SPV genome. As a specific example, in SPV kasza strain (VR-363), the TK gene is located at nt55625-56167. The exact position of the IL18bp gene in any strains of SPV may be identified easily using common knowledge, routine sequence analysis and/or sequence alignment. Preferred rSPVs of the invention further comprise an inactive TK gene, wherein the endogenous TK gene lacks at least 50 nt, preferably at least 100 nt, even more preferably at least 150 nt, at least 200 nt, at least 250 nt, at least 300 nt, further more preferably at least 400 nt, such as between 420 nt and 500 nt. Specific and preferred rSPVs of the invention contain a deletion of at least nt 100-300 of TK gene, even more preferably of at least nt70-450 of TK gene, even more preferably of at least nt60-500 of the TK gene, such as nt59-536 of the TK gene.

The ARP gene of a viral SPV DNA contains approximately 1455 bp, and is generally located at nt residues 137100-138554 of a SPV genome. As a specific example, in SPV kasza strain (VR-363), the ARP gene is located at nt137100-138554. The exact position of the IL18bp gene in any strains of SPV may be identified easily using common knowledge, routine sequence analysis and/or sequence alignment. As regards the ARP gene, in rSPVs of the invention comprising an inactive ARP gene, the endogenous ARP gene coding sequence preferably lacks at least 50 nt, preferably at least 100 nt, even more preferably at least 110 nt. Specific and preferred rSPVs of the invention contain a deletion of at least nt1150-1200 of ARP gene, even more preferably of at least nt1130-1220 of ARP gene, even more preferably of at least nt1116-1228 of the ARP gene. Larger deletions may also be performed, covering between 800 and 1300 bp of the ARP gene.

In a particular embodiment, the rSPVs of the invention contains a deletion of at least 100 nt in the serpin gene, a deletion of nt50-300 of IL18bp gene, and a deletion of at least nt70-450 of TK gene.

In a specific embodiment, the rSPV contains a deletion of nt31-382 or nt19-369 of IL18bp gene and a deletion of nt59-536 of the TK gene.

In a particular embodiment, the rSPV of the invention comprises at least one foreign nucleic acid sequence, which may be located in any non-essential position in the SPV genome, preferably within (or in place of) one of the above inactive regions.

The insertion of a foreign nucleic acid sequence in the SPV genome can be performed by known methods such as mutagenesis, PCR, homologous recombination, etc. In a particular embodiment, a shuttle vector (or insertion plasmid) is prepared by recombinant DNA technology in which a foreign nucleic acid sequence is cloned flanked by two viral homology regions. The homology regions typically contain each between 50-1000 nt of a target gene sequence, allowing specific homologous recombination. The shuttle vector may be prepared from any known or conventional plasmids, cosmids, phages, and the like, such as pBS plasmids, pBR322, pUC18, pUC19 and pHC79. Examples of shuttle vectors (or insertion plasmids) are provided in FIGS. 3 and 4. The shuttle vector may then be introduced into an SPV-infected cell using known techniques such as electroporation, calcium phosphate, a lipofectin-based method, or the like. Recombinant SPV viruses having integrated the foreign nucleic acid sequence are then selected. Their sequence can be verified. The rSPV can then be maintained in any suitable competent cell. The virus can be maintained in culture, or purified and frozen or lyophilized.

A particular rSPV of the invention comprises an inactive serpin gene and further comprises a foreign nucleic acid sequence inserted into the IL18bp gene of the rSPV genome, preferably in replacement of at least part of said gene sequence. Preferably, such particular rSPV also comprises an inactive TK gene.

Another particular rSPV of the invention comprises an inactive serpin gene and further comprises a foreign nucleic acid sequence inserted into the TK gene of the rSPV genome, preferably in replacement of at least part of said gene sequence. Preferably, such particular rSPV also comprises an inactive IL18bp gene.

Another particular rSPV of the invention comprises an inactive serpin gene and further comprises a foreign nucleic acid sequence inserted into the inactive serpin gene of the rSPV genome. Preferably, such particular rSPV also comprises an inactive IL18bp gene and/or an inactive TK gene.

Foreign Nucleic Acid Sequence

The foreign nucleic acid sequence may be any nucleic acid sequence or molecule not naturally present in a SPV genome, or not naturally present at such a location in a SPV genome. A foreign nucleic acid typically comprises a gene sequence encoding an mRNA, a peptide or a polypeptide (or protein). The foreign gene sequence may, for instance, encode various types of active molecules, such as an antigen, adjuvant, cytokine, lymphokine, growth factor, enzyme, label, etc.

In a preferred embodiment, the foreign gene sequence encodes an antigen (peptide, polypeptide or protein antigen) from a pathogen of a porcine infectious disease, and most preferably an antigen from a virus, bacterium, fungus, or protozoa. Within the context of the invention, a peptide typically designates a molecule comprising from 4 to 30 amino acids. A polypeptide is any amino acid polymer comprising more than 30 amino acids. The term polypeptide includes full length proteins.

The foreign gene sequence preferably encodes a peptide or polypeptide (e.g., glycoprotein, capsid protein, or fragment thereof) of a virus or pathogen selected from porcine circovirus (PCV1, PCV2, PCV2a, PCV2b, PCV2d, PCV3), Actinobacillus pleuropneunomia; Adenovirus; Alphavirus such as Eastern equine encephalomyelitis viruses; Balantidium coli; Bordetella bronchiseptica; Brachyspira spp., preferably B. hyodyentheriae, B. pilosicoli, B. innocens, Brucella suis, preferably biovars 1, 2 and 3; Classical swine fever virus, African swine fever virus; Chlamydia and Chlamydophila sp. and preferably C. pecorum and C. abortus; Clostridium spp., preferably Cl. difficile, Cl. perfringens types A, B and C, Cl. novyi, Cl. septicum, Cl. tetani; Digestive and respiratory Coronavirus; Cryptosporidium parvum; Eimeria spp; Eperythrozoonis suis currently named Mycoplasma haemosuis; Erysipelothrix rhusiopathiae; Escherichia coli; Haemophilus parasuis, preferably subtypes 1, 7 and 14; Hemagglutinating encephalomyelitis virus; Lsospora suis; Japanese Encephalitis virus; Lawsonia intracellulars; Leptospira spp., preferably Leptospira australis, Leptospira canicola, Leptospira grippotyphosa, Leptospira icterohaemorrhagicae, Leptospira interrogans, Leptospira Pomona and Leptospira tarassovi; Mannheimia haemolytica; Mycobacterium spp. preferably, M. avium, M. intracellular and M. bovis: Mycoplasma hyponeumoniae; Parvovirus; Pasteurella multocida; Porcine cytomegolovirus; Porcine parovirus, Porcine reproductive and respiratory syndrome virus: Pseudorabies virus; Rotavirus; Sagiyama virus; Salmonella spp. preferably, S. thyhimurium and S. choleraesuis; Staphylococcus spp. preferably, S. hyicus; Streptococcus spp., preferably Strep, suis; Swine cytomegalovirus; Swine herpes virus; Swine influenza virus; Swinepox virus; Toxoplasma gondii; Vesicular stomatitis virus or virus of exanthema of swine.

In a particularly preferred embodiment, the foreign gene sequence encodes a PCV2 antigen, particularly a PCV2 protein or peptide, even more particularly a PCV2 capsid (e.g., ORF2) protein or peptide.

In another particular embodiment, the foreign gene sequence encodes a PCV3 antigen, particularly a PCV3 protein or peptide, even more particularly a PCV3 capsid protein or peptide.

The foreign gene sequence may contain a transcriptional promoter to allow or increase expression of the encoded mRNA or polypeptide. The promoter used may be a synthetic or natural promoter, including a swinepox promoter, a poxvirus promoter, or a promoter derived from different viruses or cells such as promoters derived from eukaryotic or prokaryotic organisms. Specific examples of promoters include the vaccinia virus 7.5-kD promoter (P7.5k) (Davison A. J. et al., J. Mol. Biol., 210(4):749-69 (1989)), 11-kD promoter (P11k) (Bertholet et al., Proc. Nat. Acad. Sci., 82:2096-2100 (1985)) or 28-kD promoter (P28k) (Weir J. P. & Moss B., J. Virol. 61:75-80 (1987)), or an artificial synthetic Poxvirus promoter (Ps), the thymidine kinase promoter of herpesvirus (Ross L. J., Gen. Virol. 74:371-377 (1993)), gB protein promoter (supra) of HVT or MDV, the IE promoter of human cytomegalovirus (HCMV) (Alting-Mess M. A., Nucleic Acids Res., 17:9494 (1989)), SV40 promoter (Gunning P., Proc. Natl. Acad. Sci., 84:4931-4835 (1987)), [beta] actin promoter (supra, and Kost A. T., Nucleic Acids Res., 11:8287-8301 (1983)), [beta]-globin promoter (Spitzner J. R., Nucleic Acids Res., 18:1-11 (1990)), the LTR promoter of Rous sarcoma virus (Fiek A. et al., Nucleic Acids Res., 20:1785 (1992)), and the like. In addition, promoters of the structural proteins or the essential genes of SPV can also be used.

rSPV of the invention may contain several foreign gene sequences, located in a same cloning region and/or in distinct cloning sites.

In a particular embodiment, the rSPV of the invention comprises at least 2 foreign gene sequences encoding two distinct antigens (from a same or distinct pathogen). In this regard, in a further particular embodiment, the rSPV of the invention comprises at least a foreign gene sequence encoding a PCV2 antigen and a foreign gene sequence encoding a distinct antigen. In another particular embodiment, the rSPV of the invention comprises a foreign gene sequence encoding an antigen and a foreign gene sequence encoding an adjuvant or a cytokine.

In a further particular embodiment, the rSPV of the invention comprises at least two foreign gene sequences each encoding a PCV2 antigen, particularly each gene sequence encodes an ORF2 protein or peptide, which may be the same of different.

In multivalent rSPVs of the invention, the at least two foreign gene sequences may be under the control of the same or distinct promoters, and in the same or opposite orientation.

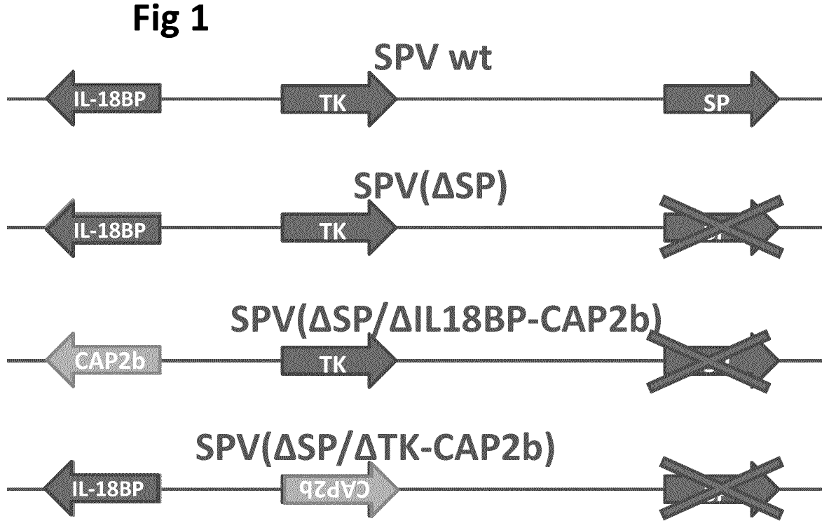

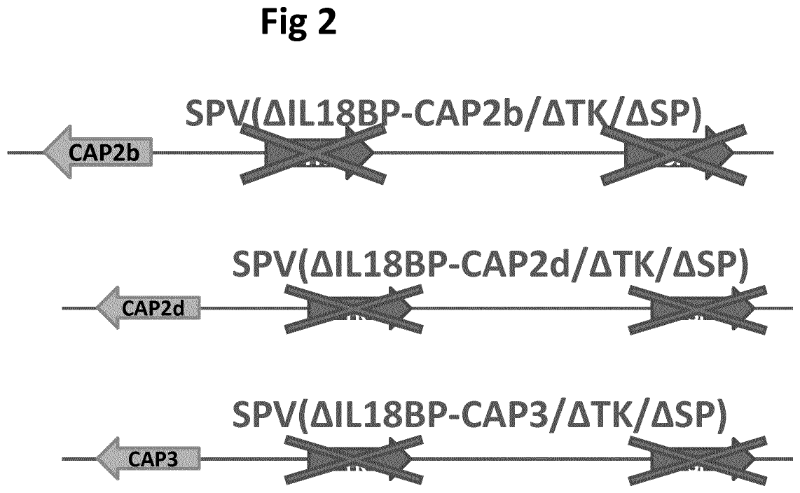

Specific examples of rSPVs of the invention are disclosed FIGS. 1 and 2.

Nucleic acid Molecules

The invention also relates to nucleic acid molecules comprising the genome of a rSPV of the invention. Nucleic acid molecules of the invention may be DNA or RNA, double-stranded or single-stranded. Single-stranded DNA or RNA may be the coding strand, also known as the sense strand, or it may be the non-coding strand, also referred to as the anti-sense strand. The invention also relates to variants or analogs of such nucleic acid molecules, e.g., molecules having at least 85%, 90%, 95%, 96%, 97%, 98% or more sequence identity thereof.

The degree of homology between two nucleic acid sequences may be determined by means of computer programs known in the art such as GAP provided in the GCG program package (Program Manual for the Wisconsin Package, Version 8, August 1996, Genetics Computer Group, 575 Science Drive, Madison, Wis., USA 5371 1) (Needleman, S. B. and Wunsch, C D., (1970), Journal of Molecular Biology, 48, 443-453). Using GAP with the following settings for DNA sequence comparison: GAP creation penalty of 5.0 and GAP extension penalty of 0.3. Nucleic acid molecules may be aligned to each other using the Pileup alignment software, available as part of the GCG program package, using, for instance, the default settings of gap creation penalty of 5 and gap width penalty of 0.3.

Suitable experimental conditions for determining whether a given nucleic acid molecule hybridizes to a specified nucleic acid may involve pre-soaking of a filter containing a relevant sample of the nucleic acid to be examined in 5×SSC for 10 minutes, and pre-hybridization of the filter in a solution of 5×SSC, 5×Denhardt's solution, 0.5% SDS and 100 [mu]g/ml of denatured sonicated salmon sperm DNA, followed by hybridization in the same solution containing a concentration of 10 ng/ml of a P-dCTP-labeled probe for 12 hours at approximately 45° C., in accordance with the hybridization methods as described in Sambrook et al. (1989; Molecular Cloning, A Laboratory Manual, 2nd edition, Cold Spring Harbour, N.Y.). The filter is then washed twice for 30 minutes in 2×SSC, 0.5% SDS at least 55° C. (low stringency), at least 60° C. (medium stringency), at least 65° C. (medium/high stringency), at least 70° C. (high stringency), or at least 75° C. (very high stringency). Hybridization may be detected by exposure of the filter to an x-ray film.

The nucleic acid molecules according to the invention may be provided in the form of a nucleic acid molecule per se such as naked nucleic acid molecules; a vector; virus or host cell etc. Vectors include expression vectors that contain a nucleic acid molecule of the invention.

Host Cells

In a further embodiment of the invention, there is provided a host cell transformed with a nucleic acid or with a rSPV according to the invention. Such cells can produce rSPVs of the invention. Suitable examples of host cells are known to those skilled in the art or can be readily selected by those skilled in the art. Host cells are preferably eukaryotic cells such as mammalian (e.g., pig), fungal (e.g. Saccharomyces cerevisiae, Pichia, Aspergillus, Fusarium), insect and plant cells. Specific examples of host cells are swine kidney cells, such as ESK-4 cells (CL-184).

Vaccine Compositions and Methods

The term “vaccine” as used herein includes any composition which may be used to cause, stimulate or amplify an immune response in an animal (e.g., pigs) against a pathogen. Particular examples of vaccines of the invention are composition able to cause or stimulate or amplify immunity against a PCV2 virus. In a vaccine of the invention, the at least one foreign gene sequence shall encode an antigen or an adjuvant.

The term “immunization” includes the process of delivering an immunogen to a subject. Immunization may, for example, enable a continuing high level of antibody and/or cellular response in which T-lymphocytes can kill or suppress the pathogen in the immunized non-human animal, such as pig, which is directed against a pathogen or antigen to which the animal has been previously exposed.

Vaccines of the invention comprise an immunologically effective amount of a rSPV or nucleic acid or cell as described above in a pharmaceutically acceptable vehicle.

In practice, the exact amount required for an immunologically effective dose may vary from subject to subject depending on factors such as the age and general condition of the subject, the nature of the formulation and the mode of administration. Appropriate “effective amount” may be determined by one of ordinary skill in the art using only routine experimentation. For instance, methods are known in the art for determining or titrating suitable dosages of a vaccine to find minimal effective dosages based on the weight of the non-human animal subject, concentration of the vaccine and other typical factors. In a typical embodiment, the vaccine comprises a unitary dose of between 10 and 10,000,000 TCID₅₀, preferably between 100 and 1,000,000 TCID₅₀, even more preferably of between 1,000 and 100,000 TCID₅₀, of a rSPV of the invention. TCID₅₀ designates the median tissue culture infective dose, i.e., the amount of virus that produces pathological change in 50% of inoculated cell cultures.

The dosage of the vaccine, concentration of components therein and timing of administering the vaccine, which elicit a suitable immune response, can be determined by methods such as by antibody titrations of sera, e.g., by ELISA and/or seroneutralization assay analysis and/or by vaccination challenge evaluation.

Vaccines may comprise other ingredients, known per se by one of ordinary skill in the art, such as pharmaceutically acceptable carriers, excipients, diluents, adjuvants, freeze drying stabilizers, wetting or emulsifying agents, pH buffering agents, gelling or viscosity enhancing additives, or preservatives, depending on the route of administration.

Examples of pharmaceutically acceptable carriers, excipients or diluents include, but are not limited to demineralised or distilled water; saline solution; vegetable based oils such as peanut oil, arachis oil, safflower oil, olive oil, cottonseed oil, maize oil, sesame oil, or coconut oil; silicone oils, including polysiloxanes, such as methyl polysiloxane, phenyl polysiloxane and methylphenyl polysolpoxane; volatile silicones; mineral oils such as light liquid paraffin oil, or heavy liquid paraffin oil; squalene; cellulose derivatives such as methyl cellulose, ethyl cellulose, carboxymethylcellulose, carboxymethylcellulose sodium salt, or hydroxypropyl methylcellulose; lower alkanols, for example ethanol or iso-propanol; lower aralkanols; lower polyalkylene glycols or lower alkylene glycols, for example polyethylene glycol, polypropylene glycol, ethylene glycol, propylene glycol, 1,3-butylene glycol or glycerin; fatty acid esters such as isopropyl palmitate, isopropyl myristate or ethyl oleate; polyvinylpyrrolidone; agar; carrageenan; gum tragacanth or gum acacia, and petroleum jelly. Typically, the carrier or carriers will form from 10% to 99.9% by weight of the vaccine composition and may be buffered by conventional methods using reagents known in the art, such as sodium hydrogen phosphate, sodium dihydrogen phosphate, potassium hydrogen phosphate, potassium dihydrogen phosphate, a mixture thereof, and the like.

Examples of adjuvants include, but are not limited to, oil in water emulsions, aluminum hydroxide (alum), immunostimulating complexes, non-ionic block polymers or copolymers, cytokines (like IL-1, IL-2, IL-7, IFN-[alpha], IFN-[beta], IFN-y, etc.), saponins, monophosphoryl lipid A (MLA), muramyl dipeptides (MDP) and the like. Other suitable adjuvants include, for example, aluminum potassium sulfate, heat-labile or heat-stable enterotoxin(s) isolated from Escherichia coli, cholera toxin or the B subunit thereof, diphtheria toxin, tetanus toxin, pertussis toxin, Freund's incomplete or complete adjuvant, etc. Toxin-based adjuvants, such as diphtheria toxin, tetanus toxin and pertussis toxin may be inactivated prior to use, for example, by treatment with formaldehyde.

Examples of freeze-drying stabilizer may be for example carbohydrates such as sorbitol, mannitol, starch, sucrose, dextran or glucose, proteins such as albumin or casein, and derivatives thereof.

Vaccines may comprise antigens from several pathogens, such as PCV2, Actinobacillus pleuropneunomia; Adenovirus; Alphavirus such as Eastern equine encephalomyelitis viruses; Balantidium coli; Bordetella bronchiseptica; Brachyspira spp., preferably B. hyodyentheriae, B. pilosicoli, B. innocens, Brucella suis, preferably biovars 1, 2 and 3; Classical swine fever virus, African swine fever virus; Chlamydia and Chlamydophila sp. and preferably C. pecorum and C. abortus; Clostridium spp., preferably Cl. difficile, Cl. perfringens types A, B and C, Cl. novyi, Cl. septicum, Cl. tetani; Digestive and respiratory Coronavirus; Cryptosporidium parvum; Eimeria spp; Eperythrozoonis suis currently named Mycoplasma haemosuis; Erysipelothrix rhusiopathiae; Escherichia coli; Haemophilus parasuis, preferably subtypes 1, 7 and 14; Hemagglutinating encephalomyelitis virus; lsospora suis; Japanese Encephalitis virus; Lawsonia intracellulars; Leptospira spp., preferably Leptospira australis, Leptospira canicola, Leptospira grippotyphosa, Leptospira icterohaemorrhagicae, Leptospira interrogans, Leptospira Pomona and Leptospira tarassovi; Mannheimia haemolytica; Mycobacterium spp. preferably, M. avium, M. intracellular and M. bovis: Mycoplasma hyponeumoniae; Parvovirus; Pasteurella multocida; Porcine cytomegolovirus; Porcine parovirus, Porcine reproductive and respiratory syndrome virus: Pseudorabies virus; Rotavirus; Sagiyama virus; Salmonella spp. preferably, S. thyhimurium and S. choleraesuis; Staphylococcus spp. preferably, S. hyicus; Streptococcus spp., preferably Strep, suis; Swine cytomegalovirus; Swine herpes virus; Swine influenza virus; Swinepox virus; Toxoplasma gondii; Vesicular stomatitis virus and/or virus of exanthema of swine.

The vaccine compositions of the invention may be liquid formulations such as an aqueous solution, water-in-oil or oil-in-water emulsion, syrup, an elixir, a tincture, a preparation for parenteral, subcutaneous, intradermal, intramuscular or intravenous administration (e.g., injectable administration), such as sterile suspensions or emulsions. Such formulations are known in the art and are typically prepared by dissolution of the antigen and other typical additives in the appropriate carrier or solvent systems. Liquid formulations also may include suspensions and emulsions that contain suspending or emulsifying agents.

The route of administration can be percutaneous, via mucosal administration, or via a parenteral route (intradermal, intramuscular, subcutaneous, intravenous, or intraperitoneal). The vaccine of the invention can conveniently be administered intranasally, transdermally (i.e., applied on or at the skin surface for systemic absorption), parenterally, ocularly, etc. The parenteral route of administration includes, but is not limited to, intramuscular, intravenous, intraperitoneal routes and the like.

The vaccines of the invention can be administered as single doses or in repeated doses. The vaccines of the invention can be administered alone, or can be administered simultaneously or sequentially administered with one or more further compositions, such as for example other porcine immunogenic or vaccine compositions. Where the compositions are administered at different times the administrations may be separate from one another or overlapping in time.

The present invention also relates to methods of immunizing or inducing an immune response in a non-human mammal (e.g., pigs) comprising administering to said mammal a rSPV or a nucleic acid, or a cell or a vaccine as described above.

Vaccines of the invention are preferably administered to pigs, adult pigs, but also to young pigs, piglets or to pregnant sow. Vaccination of pregnant sows is advantageous as it can confer passive immunity to the newborns via the transmission of maternal antibodies. The pigs may be less than 7, 6, 5, 4, 3, 2 or 1 week old; 1 to 6 weeks old; 2 to 5 weeks old; or 3 to 4 weeks old. Desirably, the vaccine is administered to a subject who has not yet been exposed to the pathogen.

The present invention also provides a container comprising an immunologically effective amount a rSPV, nucleic acid, cell or vaccine as described above. The invention also provides vaccination kits comprising an optionally sterile container comprising an immunologically effective amount of the vaccine, means for administering the vaccine to animals, and optionally an instruction manual including information for the administration of the immunologically effective amount the composition for treating and/or preventing infectious disease.

PCV Vaccine

The invention is particularly suited for the treatment (preventive curative) of PCV infection and associated diseases, particularly PCV2 infection and associated diseases.

Currently developed PCV2 vaccines, such as Circovac® (Merial), Ingelvac®, CircoFLEX (Boehringer lngelheim Vetmedica), or Suvaxyn®, are either inactivated PCV2 vaccines or Sub-Unit vaccines. PCV2 Sub-Unit vaccines typically use a purified, recombinant PCV2A capsid protein produced by recombinant expression of the ORF2 gene of PCV2A. In this regard, the protein encoded by ORF2 of PCV2 isolates Imp1011 has been reported in EP1741785. A protein encoded by ORF2 of PCV2 isolate PCV2Rm has been reported in WO2010/061000. The protein encoded by ORF2 of PCV2 isolate 412 has been reported in EP1816200. Another protein encoded by an ORF2 of a further PCV2 isolate has been reported in EP1036180 or EP2225367. Improved synthetic ORF2-type proteins have been described in WO2013/030320 and in WO2014/167060.

In a particular embodiment, the present invention relates to a rSPV as defined above wherein the foreign gene sequence encodes a PCV2 antigen, more preferably a PCV2 protein, polypeptide or peptide. In a more preferred embodiment, the present invention relates to a rSPV as defined above wherein the foreign gene sequence encodes a PCV2 ORF2 polypeptide or a fragment thereof. In a particular embodiment, the ORF2 is selected from ORF2 of PCV2 isolates Imp1011, PCV2Rm, or 412, or a ORF2 having at least 80% sequence identity to such proteins, or an immunogenic fragment thereof comprising at least 10, 15, more preferably at least 20 contiguous amino acid residues thereof.

In another particular embodiment, the present invention relates to a rSPV as defined above wherein the foreign gene sequence encodes a PCV3 antigen, more preferably a PCV3 protein, polypeptide or peptide. In a more preferred embodiment, the present invention relates to a rSPV as defined above wherein the foreign gene sequence encodes a PCV3 ORF2 polypeptide or a fragment thereof. In a particular embodiment, the ORF2 is selected from ORF2 of PCV3 isolates PCV3-US/MO2015 or a ORF2 having at least 80% sequence identity to such a protein, or an immunogenic fragment thereof comprising at least 10, 15, more preferably at least 20 contiguous amino acid residues thereof.

A further aspect of the invention relates to methods of treating and/or preventing a PCV2 associated disease in a non-human mammal, and to methods of immunizing or vaccinating a non-human animal subject, such as pigs, swine, sow, piglet, against PCV2 infection, comprising administering to said animal subject a rSPV, a nucleic acid, a cell, or vaccine composition as defined above.

A further aspect of the invention relates to methods of treating and/or preventing a PCV3 associated disease in a non-human mammal, and to methods of immunizing or vaccinating a non-human animal subject, such as pigs, swine, sow, piglet, against PCV3 infection, comprising administering to said animal subject a rSPV, a nucleic acid, a cell, or vaccine composition as defined above.

PCV2 and PCV3 infections or associated diseases include inter alia Postweaning Multisystemic Wasting Syndrome (PMWS), Porcine Dermatitis and Nephropathy Syndrome (PDNS), Porcine Respiratory Disease Complex (PRDC), reproductive disorders, granulomatous enteris, exsudative epidermitis, necrotizing lymphadenitis, and congenital tremors. Preferably, a non-human animal subject, such as pig, is protected to an extent in which one to all of the adverse physiological symptoms or effects of PCV2 or PCV3 infections are significantly reduced, ameliorated or totally prevented.

In one embodiment, the vaccine compositions of the invention are administered to a pig susceptible to or otherwise at risk for PCV2 infection to enhance the subject own immune response capabilities.

Preferably, the subject is a pig which is in need of vaccination against Postweaning Multisystemic Wasting Syndrome (PMWS) and/or Porcine Dermatitis and Nephropathy Syndrome (PDNS).

Further aspects and advantages of the invention shall be disclosed in the following experimental section, which illustrates the claimed invention.

### EXAMPLES

SPV kasza strain (VR-363) and embryonic swine kidney cell, ESK-4 cells (CL-184) could be purchased from the American Type Culture Collection (ATCC). The ESK-4 cells are routinely cultured at 37° C. in 5% CO2 in Ham's F-12K medium (Gibco, Cat. No.: 21127-022) supplemented with 1% streptomycin-penicillin (Gibco, Cat. No.: 15140-122) and 5% FBS (Gibco, Cat. No.: 10437-028). For SPV genomic DNA preparation, confluent ESK-4 cells in a 225 cm2 flask can be infected with SPV and incubated for 6 days until the cells show 100% cytopathic effect (CPE). The infected cells can then be harvested by scraping the cells into the medium and centrifuging at 1300 rpm for 5 min. The medium is decanted, and the cell pellet is gently resuspended in 2 ml Phosphate Buffer Saline (PBS: 1.5 g Na2HPO4, 0.2 g KH2PO4, 0.8 g NaCl and 0.2 g KCl per litter H2O) and subjected to two successive freeze-thaws. Cellular debris are then removed by centrifuging at 3000 rpm for 5 min at 4° C. SPV virions, present in supernatant, are then pelleted by centrifugation at 20,000×g for 20 min at 4° C. The resultant pellets are then suspended with 10 mM Tris pH7.5. SPV genomic DNAs are then extracted from the SPV virions by suspending with the lysis buffer (20 mM Tris, pH9, 0.1M NaCl2, 5 mM EDTA, 0.1% SDS, 0.2 mg/ml proteinase K) and incubating at 60° C. for 5 min. Phenol:chlororoform (1:1) extraction is conducted two times, and the sample precipitated by the addition of two volumes of ethanol and centrifugation. The supernatant is decanted, and the pellet (SPV DNA) is air dried and rehydrated in 10 mM Tris pH7.5, 1 mM EDTA at 4° C.

The flanking regions of serpin (SP) gene in the SPV genome were cloned by Polymerase Chain Reaction (PCR). The amplified DNA fragment was cloned into a plasmid and ampicillin-resistant transformants were picked up and grown in LB broth containing 50 micro-g/ml ampicillin. One of the candidate plasmids, pD-SP, was used as a basic insertion plasmid (FIG. 4).

Recombinant SPVs are generated in ESK-4 cells by homologous recombination between wild-type SPV genome and insertion plasmid vectors. Sub-confluent ESK-4 cells in a 6-well plate are infected with wild-type SPV (wtSPV) or with a recombinant SPV having an inactive IL18bp gene and/or an inactive TK gene, and 17 hr later the infected cells are transfected with 2 μg of pD-SP using Lipofectamin Plus reagent (Invitrogen) and allowed to incubate at 37° C. for 5 days until cytopathic effect (CPE) has occurred. Cell lysates from infected-transfected cells can be collected and transferred to new blank 96-well plates, and infected cells lysed with lysis buffer (20 mM Tris-Cl, 0.1M NaCl, 5 mM EDTA, 0.1% SDS, 200 μg/ml protenase K) followed by heat treatment (60° C. 5 min, and 98° C. 2 min). The rSPVs are isolated from said lysate. The genomic structure of different rSPVs is represented FIGS. 1 and 2.

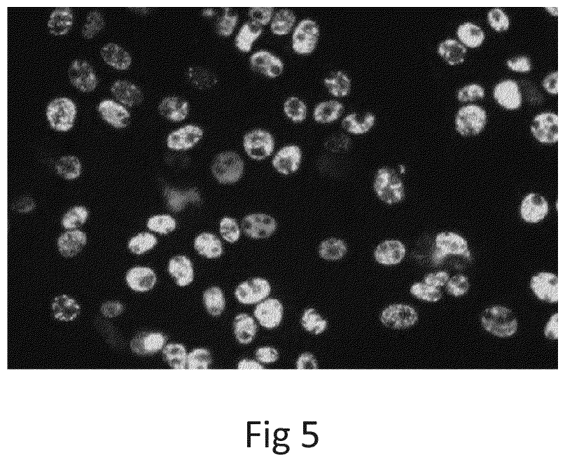

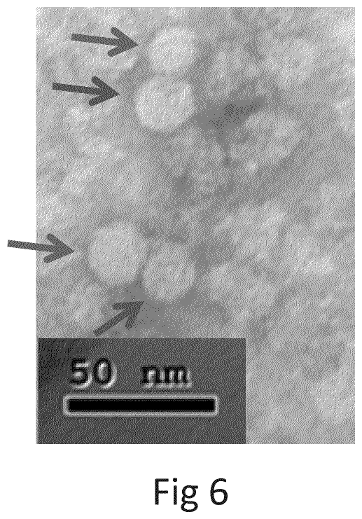

PCV2-ORF2 proteins expressed by recombinant SPVs are analyzed. ESK-4 cells in 6-well plates are infected with rSPVs at a multiplicity of infection (M.O.I.) of 0.1. Six days later, cell lysates are analyzed, showing PCV2 nuclear expression and VLP-type particles (FIGS. 5 and 6).

1-18. (canceled)

19. A recombinant swinepox virus (rSPV) comprising an inactive serpin gene.

20. The rSPV of claim 19, further comprising an inactive thymidine kinase (TK) gene and/or an inactive IL18bp gene.

21. The rSPV of claim 19, comprising an inactive serpin gene, an inactive thymidine kinase (TK) gene, and an inactive IL18bp gene.

22. The rSPV of claim 19, wherein the rSPV genome comprises a deletion of at least 50 bp or at least 100 bp in the coding sequence of each inactive gene.

23. The rSPV of claim 19, said rSPV comprising at least one foreign nucleic acid sequence.

24. The rSPV of claim 23, wherein the foreign nucleic acid sequence is inserted in a region of the rSPV genome selected from the IL18bp gene, the TK gene, the serpin gene, or the Ankyrin repeat protein gene.

25. The rSPV of claim 23, wherein the at least one foreign nucleic acid sequence encodes an antigen.

26. The rSPV of claim 25, wherein the foreign nucleic acid sequence encodes a porcine circovirus (PCV) antigen, a PCV2 antigen, a PCV2 capsid antigen, or a PCV2 ORF2 protein or peptide.

27. The rSPV of claim 23, wherein the foreign nucleic acid sequence contains a transcriptional promoter.

28. The rSPV of claim 27, wherein the promoter is selected from the vaccinia virus 7.5-kD promoter (P7.5k), 11-kD promoter (P11k), or 28-kD promoter (P28k), an artificial synthetic Poxvirus promoter (Ps), the chicken beta-actin (Bac) promoter or a derivative thereof, the Pec promoter, the Murine Cytomegalovirus (Mcmv) immediate-early (ie)1 promoter, the Human Cytomegalovirus promoter (Hcmv), the Simian virus (SV)40 promoter, and the Raus Sarcoma virus (RSV) promoter, or fragments thereof which retain a promoter activity.

29. The rSPV of claim 19, which comprises an inactive serpin gene and which further comprises a nucleic acid sequence encoding a PCV antigen inserted into the IL18bp gene of the rSPV genome.

30. A nucleic acid molecule comprising the genome of a rSPV of claim 19.

31. A host cell comprising a rSPV of claim 19 or a nucleic acid molecule encoding the genome of said rSPV.

32. A method for producing a rSVP comprising infecting a competent cell with a nucleic acid molecule of claim 30 and collecting the rSVP.

33. A composition comprising a rSVP of claim 19 and an excipient.

34. The composition of claim 33, said composition comprising a vaccine composition.

35. A method of immunizing a porcine comprising administering a rSPV of claim 19 to said porcine.

36. A vaccination kit for immunizing a porcine which comprises the following components:
a) an effective amount of a vaccine composition of claim 34, and
b) a means for administering said vaccine to said porcine.
